(19) United States
(12) Patent Application Publication
(10) Pub. No.: US 2026/0224859 A1
(43) Pub. Date: Aug. 6, 2026

(54) MEDICAL GUIDE POSITIONING DEVICE

(71) Applicant: CHINESE ACADEMY OF MEDICAL SCIENCES FUWAI HOSPITAL SHENZHEN HOSPITAL (SHENZHE SUN YAT-SEN CARDIOVASC, Shenzhen (CN)

(72) Inventor: Xiangbin PAN, Shenzhen (CN)

(73) Assignee: CHINESE ACADEMY OF MEDICAL SCIENCES FUWAI HOSPITAL SHENZHEN HOSPITAL (SHENZHE SUN YAT-SEN CARDIOVASC, Shenzhen (CN)

(21) Appl. No.: 19/155,957

(22) PCT Filed: Jun. 30, 2023
(86) PCT No.: PCT/CN2023/104383

§ 371 Date: Aug. 13, 2025

(51) Int. Cl. A61M 25/09; A61B 17/22

(52) U.S. Cl. CPC A61M 25/09041; A61B 2017/22038; A61M 2205/0216; A61M 2210/12

(57) ABSTRACT

The provided is a medical guide positioning device, including: a guide positioning mechanism, the guide positioning mechanism including a resilient positioning part and an extension part; the resilient positioning part is a hollow mesh body, with the front end portion thereof being a curved surface protruding in the direction of the direction of backing away from the extension part, and the connection between the resilient positioning part and the extension part is a joint, the resilient positioning part having an initial state and a deformation state; the extension part has an opposing first end and a second end, the proximity of the elastically positioned portion is the first end, and between the first end and the second end are a number of lengths of lead wires. The provided solves the problems of inconvenient positioning, low protection performance and poor overall practicability of the existing technology.

### CROSS REFERENCE TO THE RELATED APPLICATIONS

This application is the national phase entry of International Application No. PCT/CN 2023/104383, filed on Jun. 30, 2023, the entire contents of which are incorporated herein by reference.

### TECHNICAL FIELD

The present invention relates to the technical field of medical devices, specifically, to a medical guide positioning device.

### BACKGROUND

Currently, some surgeries require the insertion of a long-sized medical instrument, such as a catheter, from the rest of the lower limb, arm or wrist, and then a guidewire is inserted into the catheter to be delivered to the lesion site for treatment.

However, conventional guidewires are inconvenient to position, have low protective properties, are prone to damage the internal tissues of the patient, and have an overall low utility that fails to meet the needs of the industry. For example, in the invention patent with patent number CN 202080022376.6, filed on Mar. 10, 2020, and titled guidewire, the visual recognition is improved by setting different changing patterns on the guidewire to facilitate the precise control of the position of the guidewire during the operation, but the protection of the guidewire has not been improved, and the front portion of the guidewire is prone to cause damage to the internal tissues of the human body, increasing the risk of surgery. However, it does not improve the protection of the guidewire, and the front part of the guidewire is easy to cause damage to the internal tissues of the human body, which increases the risk of surgery.

### SUMMARY

The purpose of the present invention is to provide a medical guide positioning device, in order to solve the problems of inconvenient positioning, low protection performance and poor overall practicability of the existing technology.

To solve the above problems, the present invention firstly provides a medical guide positioning device comprising: a guide positioning mechanism, the guide positioning mechanism comprising a resilient positioning part and an extension part; the resilient positioning part is a hollow mesh body, the front end portion of which is a curved surface protruding in the direction of the direction of departing from the extension part, the connection between the resilient positioning part and the extension part is a connection point, and the resilient positioning part has an initial state and a deformation state; the extension part has a first end and a second end opposite to each other, the one near the elastic positioning part is the first end, and between the first end and the second end are a number of lengths of lead wires; in the initial state, the point on the elastic positioning part that is furthest from the connection point is the distal end point, and both the connection point and the distal end point are located in the axis of the elastic positioning part, and the first end or a point between the first end and the second end is the connection point, and the distance between the first end and the connection point is less than a distance between the distal end point The distance between the first end and the connection point is less than the distance between the distal end point and the connection point; and when in the deformed state, the resilience of the resilient positioning portion is configured to support movement of the first end to any point on the outer surface of the resilient positioning portion.

The elastic positioning portion can be deformed at the front end of the guiding and positioning mechanism, the extension portion is connected to the elastic deformation portion, and the elasticity thereof is configured to support the movement of the first end to any point on the outer surface of the elastic deformation portion, so that the elastic positioning portion can be changed at will during the transmission process, thereby accelerating the efficiency of the transmission; furthermore, by setting the front end of the elastic positioning portion as a curved surface protruding in the direction of the direction of the backward extension portion, the protection performance is strengthened. Making the elastic positioning portion have a larger force area when it reaches the inside of the human tissue, avoiding damage; additionally setting the first end or a point between the first end and the second end as a connecting point, and the distance between the first end and the connecting point is less than the distance from the distal end point to the connecting point, so that the first end is a connecting point or is located in the hollow interior of the elastic positioning portion, which facilitates the formation of different morphing shapes and is suitable for surgeries in different situations, and Enhancing the applicability of the device; in addition, the hollow structure of the mesh facilitates conveying ultrasound signals, which can strengthen the positioning function and optimize the visibility of the guided positioning mechanism during the transmission process, and the device has been improved in a number of aspects, which overall enhances the practicality of the medical guided positioning device.

Further, in the initial state, the resilient positioning portion includes an outer surface and an inner surface that are smoothly connected and spaced apart, the outer surface and the inner surface are both curved surfaces that project in a direction away from the extension portion, and a surface area of the inner surface is smaller than a surface area of the outer surface.

Further, in the initial state, the resilient positioning portion includes a connected outer convex portion and a curved edge portion, the outer convex portion includes spaced outer and inner curved surfaces, the surface area of the inner curved surface is smaller than the surface area of the outer curved surface, the outer and inner curved surfaces are protruding in the direction of the direction of the extension portion, and the curved edge portion curves in a direction perpendicular to the extension length direction, and the curved edge portion includes a first curved surface and a second curved surface smoothly connected and spaced apart. The first surface is connected to an end portion of the inner curved surface, and the second surface is connected to an end portion of the outer curved surface.

Further, in the initial state, the elastic positioning portion includes a first mesh body and a second mesh body connected, the second mesh body having a volume smaller than the first mesh body and located in an inner cavity of the first mesh body, the first mesh body and the second mesh body both having spaced apart and smoothly connected outer curved surfaces and inner curved surfaces, the inner curved surfaces having a surface area smaller than the surface area of the outer curved surfaces, and the outer curved surfaces and the inner curved surfaces being protruding in a direction away from the extension portion. protruding.

Further, in the initial state, the elastic positioning portion includes at least two spheres of different diameters, and the spherical centers of the plurality of spheres are located in the same straight line with the connection point.

Further, in the initial state, the shape of the resilient positioning portion is one of a sphere, an ellipsoid, or an ovoid.

Further, when in the initial state, the resilient positioning portion includes a first arcuate surface at a front portion and a second arcuate surface connected to the extension portion, the first arcuate surface and the second arcuate surface being connected to each other by a third arcuate surface, and a projection direction of the second arcuate surface being in the opposite direction of the projection direction of the first arcuate surface.

Further, a conduit and a loading mechanism are included, the loading mechanism being a hollow tubular shape having an opposing connecting end and a loading end, the connecting end being coupled to an end of the conduit, and the guiding and positioning mechanism entering into the loading mechanism from the loading end.

Further, a notch is provided in a side wall between the connecting end and the loading end, the notch's being configured to support passage of an extension.

Further, the loading mechanism includes a smooth section and an outwardly expanding section connected sequentially from front to back, the smooth section having a uniform tube diameter and the outwardly expanding section having a tube diameter that gradually increases from front to back.

### BRIEF DESCRIPTION OF THE DRAWINGS

In order to more clearly illustrate the technical solutions in the embodiments of the present invention or the prior art, the following will briefly introduce the accompanying drawings that need to be used in the description of the embodiments or the prior art, and it is obvious that the accompanying drawings in the following description are only the embodiments of the present invention, and for the person of ordinary skill in the field, other attachments can be obtained based on the accompanying drawings provided, without paying creative labor. FIG. 1 shows an embodiment of the present invention.

FIG. 1 shows a schematic structural diagram of a medical guide positioning device provided by an embodiment of the present invention;

FIG. 2 shows a schematic diagram of the initial state structure of the guide positioning mechanism provided by the embodiment of the present invention (I);

FIG. 3 shows a schematic diagram of the initial state structure of the guide positioning mechanism provided by the embodiment of the present invention (II);

FIG. 4 shows a schematic diagram of the initial state structure of the guided positioning mechanism provided by the embodiment of the present invention (III);

FIG. 5 shows a schematic diagram of the initial state structure of the guided positioning mechanism provided by the embodiment of the present invention (IV);

FIG. 6 shows a schematic diagram of the initial state structure of the guided positioning mechanism provided by the embodiment of the present invention (V);

FIG. 7 shows a schematic diagram of the initial state structure of the guided positioning mechanism provided by the embodiment of the present invention (VI);

FIG. 8 shows a schematic diagram of the deformation state structure of the guided positioning mechanism provided by the embodiment of the present invention;

FIG. 9 shows a schematic diagram of the initial state structure of the guided positioning mechanism provided by the embodiment of the present invention (VII);

FIG. 10 shows a schematic diagram of the initial state structure of the guided positioning mechanism provided by the embodiment of the present invention (viii).

Description of the Accompanying Drawings Markings
100—guided positioning mechanism; 110—elastic positioning portion; 111—connection point; 112—distal end point; 113—external convex portion; 1131—external curved surface; 1132—internal curved surface; 114—bent edge portion; 1141—first curved surface; 1142—second curved surface; 115—first curved surface; 116—second curved surface; 117—third curved surface; 120—extension portion; 121—first end; 122—second end;
200—conduit;
300—loading mechanism; 301—connecting end; 302—loading end; 303—notch; 310—smoothing section; 320—expanding section.

### DETAILED DESCRIPTION OF THE EMBODIMENTS

In order to make the above objects, features and advantages of the present invention more obvious and understandable, the specific embodiments of the present invention are described in detail below in conjunction with the accompanying drawings. It should be understood that the specific embodiments described herein are only for explaining the present invention and are not intended to limit the present invention.

### Example 1

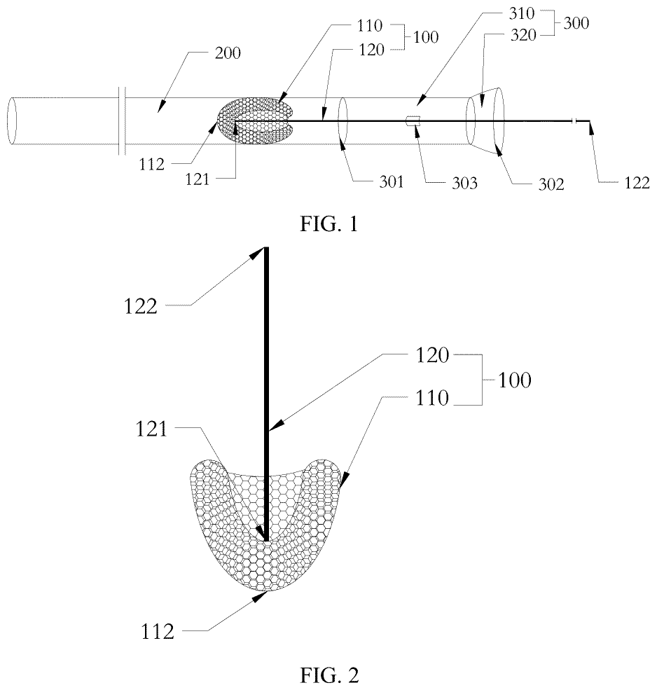

As the existing medical guide positioning device has the problems of inconvenient positioning, low protection performance and poor overall practicability, in order to solve the above problems, the present embodiment firstly provides a medical guide positioning device, as shown in FIG. 1, comprising a guide positioning mechanism 100, a catheter 200, and a loading mechanism 300, wherein the loading mechanism 300 is a hollow tubular shape, having an opposing connecting end 301 and loading end 302, the connecting end 301 is connected to the end of the catheter 200, and the guide positioning mechanism 100 enters into the loading mechanism 300 from the loading end 302.

Specifically, the guide positioning mechanism 100 includes a resilient positioning section 110 and an extension section 120; the resilient positioning section 110 is a hollow mesh body, the front end of which is a curved surface protruding in a direction away from the extension section 120, and the connection between the resilient positioning section 110 and the extension section 120 is a connection point 111, and the resilient positioning section 110 has an initial state and a deformation state; the extension section 120 has a first end 121 and a second end 122 that are opposite end 121 and a second end 122, the one near the elastically positioned portion 110 is the first end 121, and between the first end 121 and the second end 122 are a number of lengths of lead wires; when in the initial state, the point on the elastically positioned portion 110 that is furthest away from the connection point 111 is the distal end point 112, and both the connection point 111 and the distal end point 112 are located in an axis of the elastically positioned portion 110, the first end 121 or a point between the first end 121 and the second end 122 is the connection point 111, and the distance between the first end 121 and the connection point 111 is less than the distance from the distal end point 112 to the connection point 111; and in the deformed state, the resilience of the resiliently positioned portion 110 is configured to support the movement of the first end 121 to any point on the outer surface of the resiliently positioned portion 110. Among other things, this embodiment lists a variety of shapes of the resilient positioning portion 110 in FIGS. 2-FIGS. 10, respectively, which are expanded upon in the following embodiments, and the traits of the resilient positioning portion 110 of the present invention include, but are not limited to, the shapes of FIGS. 2-FIGS. 10.

The present device can be applied in transcatheter 200 aortic valve replacement, and the other devices mentioned below are conventional devices in the field, which are fully comprehensible to those skilled in the field, and again will not be repeated, and the following explanations are mainly intended to reflect the referenced scenarios and effects of the present device. Currently, in this type of surgery, when the stent valve reaches the aortic annulus, the outer sheath of the delivery system can be slowly withdrawn by controlling the knob of the delivery system, and the stent valve compressed directly in the inner and outer sheaths gradually opens up due to the elasticity, returns to its natural state, and is fixed in the aortic annulus. However, due to the great tension in the delivery system during this process, and the stent valve still slides forward during the opening process, the stent valve is prone to deviate from its original position resulting in surgical failure. In contrast, the device in this embodiment can assist in stent valve positioning by inserting the elastic positioning portion 110 of the device into the left ventricle through the aortic valve, and after the valve delivery system is inserted into the human body along the device and reaches the left ventricle, conventional positioning is performed so that the bottom of the stent valve is located at the aortic annulus, and the beginning of the posterior withdrawal of the outer sheath is started, so that the distal point 112 and the nearby portion of the elastic positioning portion 110 of the device will be pushed up under the Under the aortic valve annulus, the stent valve will fall into the elastic positioning portion 110 during the gradual release process, and the curved surface of the mesh body thereof will play a supportive and protective role thereof, and at this time, the head of the delivery system will also naturally enter into the groove of the head of the present device. In the process of releasing the stent valve, by pulling the extension portion 120 of the device tightly, the elastic positioning portion 110 will play a supporting role for the stent valve, which can prevent the stent valve from sliding and play an accurate positioning effect.

In this embodiment, the front end of the guide positioning mechanism 100 is provided with an elastic positioning portion 110 that can be deformed, the extension portion 120 is connected to the elastic deformation portion, and the elastic support first end 121 is moved to any point on the outer surface of the elastic deformation portion, so that the elastic positioning portion 110 can be changed randomly during the transmission process to accelerate the efficiency of the transmission, and the front end of the elastic positioning portion 110 is provided with a curved surface protruding in the direction of the extension portion 120. 120, which strengthens the protective performance, makes the elastic positioning portion 110 have a larger force area when it reaches the inside of the human body tissues, and avoids causing damage; in addition, the first end 121 or a point between the first end 121 and the second end 122 is the connection point 111, and the distance between the first end 121 and the connection point 111 is less than the distance between the distal end point 112 and the connection point 111, which So that the first end 121 is the connection point 111 or is located inside the hollow interior of the elastic positioning section 110, which facilitates the formation of different deformation shapes applicable to surgery in different situations, and enhances the applicability of the device; in addition, the mesh-like hollow structure facilitates the conveyance of ultrasound signals, which can enhance the positioning function, and optimize the visibility of the guiding and positioning mechanism 100 in the process of transmission, and the present device is improved in several aspects, and on the whole The device is improved in several aspects, which overall enhances the utility of the medical guide positioning device.

Preferably, as shown in FIG. 1, a notch 303 is opened on the side wall of the loading mechanism 300, the extension portion 120 is able to pass through the notch 303, and the loading mechanism 300 comprises a smooth section 310 and an outwardly expanding section 320 connected sequentially from front to back, with the pipe diameter of the smooth section 310 being consistent and the pipe diameter of the outwardly expanding section 320 gradually increasing from front to back. In this way, when inserting the guide positioning mechanism 100, the extension portion 120 is first extended from the loading end 302 and pulled out from the notch 303, so as to pull the elastic positioning portion 110 to enter the smooth section 310 from the outer expanded section 320, and then be further transmitted into the conduit 200, which solves the problem that the elastic positioning portion 110 is inconveniently to enter into the conduit 200 due to its elasticity being softer and speeds up the transmission efficiency. In this embodiment, the elastic positioning portion 110 has a soft elasticity.

Among them, the elastic positioning portion 110 of the present embodiment may be provided in a variety of shapes, which are respectively described in the following embodiments.

### Example 2

As shown in FIG. 2, the resilient positioning portion 110 of the present embodiment is approximately umbrella-shaped, specifically, when in an initial state, the resilient positioning portion 110 includes an outer surface and an inner surface that are smoothly connected and spaced apart, the outer surface and the inner surface are both curved surfaces protruding in the direction away from the extension portion 120, the surface area of the inner surface is smaller than that of the outer surface, and the first end 121 is the connection point 111.

### Embodiment 3

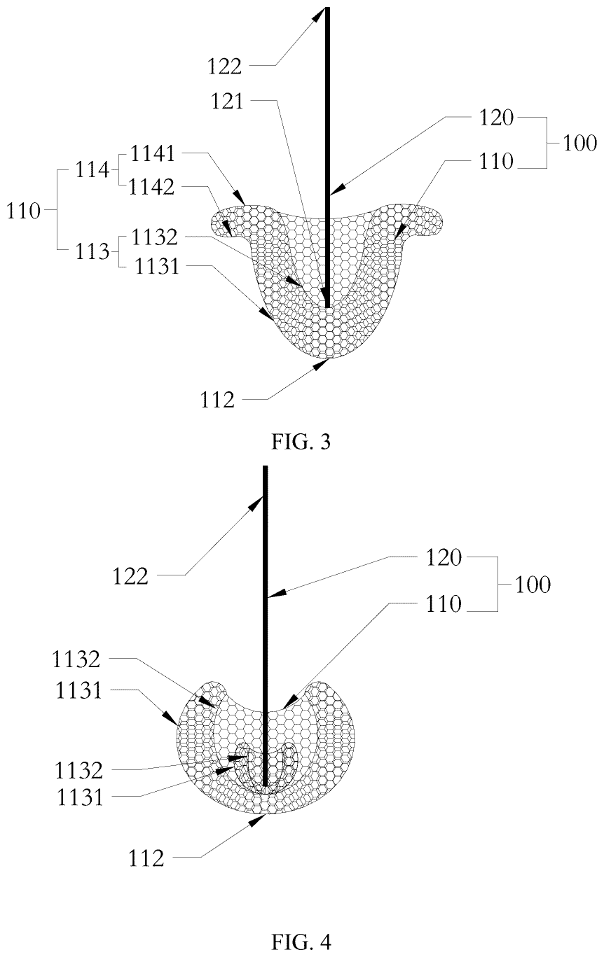

As shown in FIG. 3, the resilient positioning portion 110 of this embodiment is sub-hat-shaped and includes a connected outer convex portion 13 and a curved edge portion 114, the outer convex portion 13 includes spaced-apart outer arcuate surfaces 1131 and inner arcuate surfaces 1132, the inner arcuate surfaces 1132 have a surface area less than the surface area of the outer arcuate surfaces 1131, the outer arcuate surfaces 1131 and the inner arcuate surfaces 1132 are both projecting in the direction of backing away from the extension portion 120, and the curved edge portion 114 curves along a direction perpendicular to the length of the extension portion 120, the curved edge portion 114 includes smoothly connected and spaced apart first curved surfaces 1141 and second curved surfaces 1142, the first curved surfaces 1141 being connected to an end portion of the inner arcuate surface 1132, and the second curved surfaces 1142 being connected to an end portion of the outer arcuate surface 1131, and the first end 121 being a connection point 111 in the figure.

### Embodiment 4

As shown in FIG. 4, the resilient positioning portion 110 of this embodiment is a stacked mesh body structure, the resilient positioning portion 110 includes a connected first mesh body and a second mesh body, the second mesh body has a volume smaller than the first mesh body and is disposed in an inner cavity of the first mesh body, the first mesh body and the second mesh body both have spaced apart and smoothly connected outer arcuate surfaces 1131 and inner arcuate surfaces 1132, the inner arcuate surfaces 1132 having a surface area is less than the surface area of the outer arcuate surface 1131, and both the outer arcuate surface 1131 and the inner arcuate surface 1132 project in a direction departing from the extension portion 120, with the first end 121 in the figure being the connection point 111.

### Embodiment 5

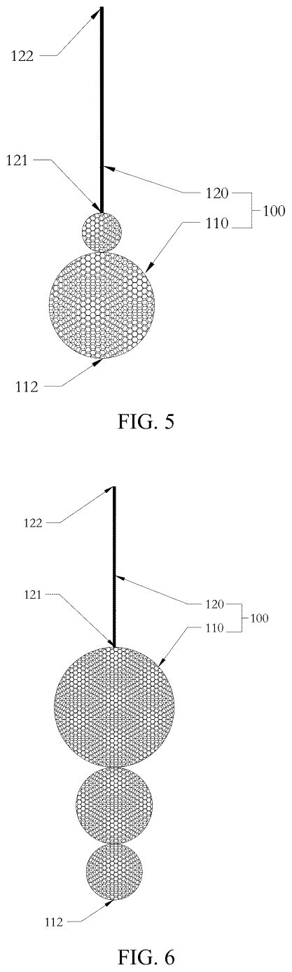

As shown in FIG. 5, the resilient positioning portion 110 of this embodiment is gourd-shaped, and when in an initial state, the resilient positioning portion 110 comprises two spheres with different diameters, the two spheres are lined up along the direction of backing away from the first end 121 in the order of the diameters from smallest to largest, and the centers of a plurality of spheres are located in the same straight line with the connection point 111, and the first end 121 is the connection point 111.

### Example of Implementation 6

As shown in FIG. 6, the resilient positioning portion 110 The resilient positioning portion 110 comprises three spheres of different diameters, the three spheres are arranged in the direction near the first end 121 in a row of diameters from small to large, and the spherical centers of the three spheres are located in the same straight line with the connection point 111. This shape includes a plurality of spheres of different diameters to facilitate measurement of the diameter of the atrial septal defect.

### Embodiment 7

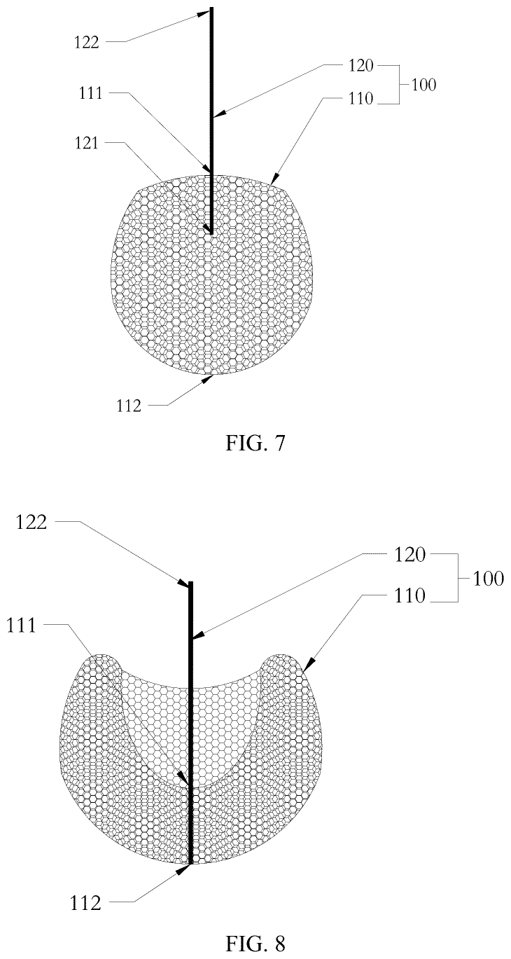

As shown in FIG. 7, the resilient positioning portion 110 of this embodiment is near-apple shaped, and when in an initial state, the shape of the resilient positioning portion 110 is ovoid, the first end 121 extends into the interior of the resilient positioning portion 110, and a point between the first end 121 and the second end 122 is the connecting point 111; as shown in FIG. 8, the schematic diagram of a deformed state of the resilient positioning portion 110 of this shape is shown in the figure, in which the first end 121 and the distal end point 112 coincide.

### Example 8

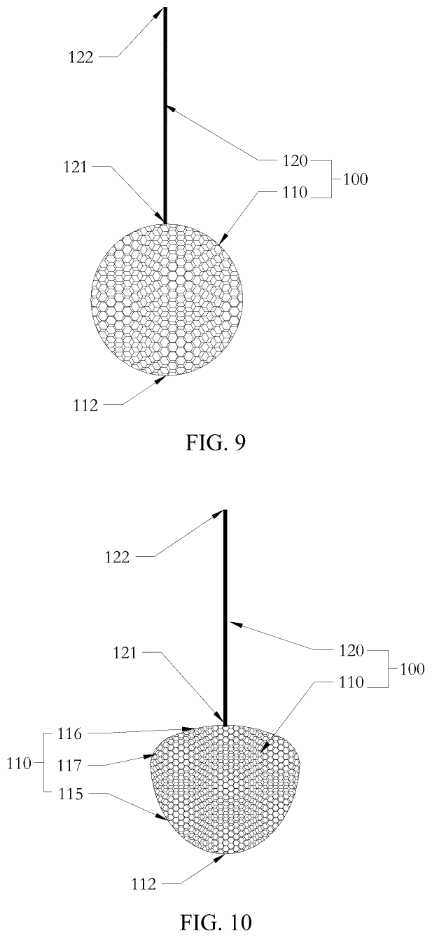

As shown in FIG. 9, in an initial state, the elastic positioning portion 110 of this embodiment is set in a spherical shape, and the first end 121 is the connection point 111.

### Example 9

As shown in FIG. 10, in the initial state, the resilient positioning portion 110 of the present embodiment is set in a subcardioid shape including a first arcuate surface 115 at a front portion and a second arcuate surface 116 connected to the extension portion 120, the first arcuate surface 115 and the second arcuate surface 116 are connected to each other by a third arcuate surface 117, and the projection direction of the second arcuate surface 116 is in the opposite direction of the projection of the first arcuate surface 115. direction opposite to that of the first arcuate surface 115.

Although the present invention is disclosed as above, the present invention is not limited thereto. Any person skilled in the art may make various changes and modifications without departing from the spirit and scope of the present invention, and therefore the scope of protection of the present invention shall be subject to the scope defined by the claims.

Finally, it should also be noted that, in this document, relationship terms such as first and second are used only to distinguish one entity or operation from another entity or operation, and do not necessarily require or imply that any such actual relationship or order exists between these entities or operations. Furthermore, the terms “including”, “comprising”, or any other variant thereof, are intended to cover non-exclusive inclusion, such that a process, method, article, or apparatus comprising a set of elements includes not only those elements, but also other elements not expressly listed, or other elements that are not expressly listed for the purpose of such a process, method, article or apparatus, or other elements that are not expressly listed for the purpose of such a process, method, article or equipment. elements, or also includes elements that are inherent to such process, method, article or apparatus. Without further limitation, the fact that an element is defined by the statement “including a . . . ” does not exclude the existence of another identical element in the process, method, article or apparatus including the said element.

Each embodiment in this specification is described in a progressive manner, and each embodiment focuses on the differences from the other embodiments, and the similarities and similarities between each embodiment can be referred to each other.

The above description of the disclosed embodiments enables those skilled in the art to realize or use the present invention. Various modifications to these embodiments will be apparent to those skilled in the art, and the general principles defined herein can be realized in other embodiments without departing from the spirit or scope of the present invention. Accordingly, the present invention will not be limited to these embodiments shown herein, but will be subject to the widest scope consistent with the principles and novel features disclosed herein.

What is claimed is:

1. A medical guide positioning device, comprising: a guide positioning mechanism, the guide positioning mechanism comprising an elastic positioning part and an extension portion;
wherein the elastic positioning part is a hollow mesh body, a front end portion of the elastic positioning part is a curved surface protruding in a direction of departing from the extension portion, a connection between the elastic positioning part and the extension portion is a connection point, and the elastic positioning part has an initial state and a deformed state;
the extension portion having opposite first and second ends, proximate the elastic positioning part being the first end, the first end being a number of lengths of lead between the first end and the second end;
when in the initial state, a point on the elastic positioning part which is furthest from a point of connection is a distal point, and both the point of connection and the distal point are located on an axis of the elastic positioning part, and the first end, or a point between the first end and the second end, is the point of connection, and a distance between the first end and the point of connection is less than a distance between the distal point and the point of connection;
when in the deformed state, resilience of the elastic positioning part is configured to support movement of the first end to any point on an outer surface of the elastic positioning part.

2. The medical guide positioning device according to claim 1, wherein when in the initial state, the elastic positioning part comprises an outer surface and an inner surface smoothly connected and spaced apart, the outer surface and the inner surface both being curved surfaces projecting in a direction of the extension portion, a surface area of the inner surface being less than a surface area of the outer surface.

3. The medical guide positioning device according to claim 1, wherein in the initial state, the elastic positioning part comprises a connected outer convex portion and a curved edge portion, the outer convex portion comprising a spaced apart outer curved surface and an inner curved surface, the inner curved surface having a surface area smaller than a surface area of the outer curved surface, both the outer curved surface and the inner curved surface projecting in a direction back from the extension portion, the curved edge portion curved along a direction perpendicular to a length of the extension portion, the curved edge portion comprising smoothly connected and spaced apart first and second curved surfaces, the first curved surface being connected to an end portion of the inner curved surface, and the second curved surface being connected to an end portion of the outer curved surface.

4. The medical guide positioning device according to claim 1, wherein in the initial state, the elastic positioning part comprises a connected first mesh body and a second mesh body, the second mesh body having a volume smaller than a volume smaller of the first mesh body and being located in an inner cavity of the first mesh body, the first mesh body and the second mesh body each having a spaced apart and smoothly connected outer curved surface and an inner curved surface, the inner curved surface having a surface area less than a surface area of the outer curved surface, the outer curved surface and the inner curved surface both projecting in a direction departing from the extension portion.

5. The medical guide positioning device according to claim 1, wherein when in the initial state, the elastic positioning part comprises at least two spheres of different diameters, spherical centers of the at least two spheres being located in an identical straight line with the connection point.

6. The medical guide positioning device according to claim 1, wherein when in the initial state, the elastic positioning part has a shape of one of a sphere, an ellipsoid and an ovoid.

7. The medical guide positioning device according to claim 1, wherein when in the initial state, the elastic positioning part comprises a first arcuate surface at a front end portion and a second arcuate surface connected to the extension portion, the first arcuate surface and the second arcuate surface being connected to each other by a third arcuate surface, and the second arcuate surface having a protruding direction opposite to a protruding direction of the first arcuate surface.

8. The medical guide positioning device according to claim 1, further comprising a conduit and a loading mechanism, the loading mechanism being a hollow tubular shape having an opposite connecting end and a loading end, the opposite connecting end being coupled to an end of the conduit, the guide positioning mechanism entering into the loading mechanism from the loading end.

9. The medical guide positioning device according to claim 8, wherein notch is opened in a side wall between the opposite connecting end and the loading end, the notch being configured to support passage of the extension portion.

10. The medical guide positioning device according to claim 9, wherein the loading mechanism comprises a smooth section and an outwardly expanding section connected sequentially from front to back, the smooth section having a uniform diameter of tube, and the outwardly expanding section having a gradually increasing diameter of tube from front to back.

11. The medical guide positioning device according to claim 2, further comprising a conduit and a loading mechanism, the loading mechanism being a hollow tubular shape having an opposite connecting end and a loading end, the opposite connecting end being coupled to an end of the conduit, the guide positioning mechanism entering into the loading mechanism from the loading end.

12. The medical guide positioning device according to claim 3, further comprising a conduit and a loading mechanism, the loading mechanism being a hollow tubular shape having an opposite connecting end and a loading end, the opposite connecting end being coupled to an end of the conduit, the guide positioning mechanism entering into the loading mechanism from the loading end.

13. The medical guide positioning device according to claim 4, further comprising a conduit and a loading mechanism, the loading mechanism being a hollow tubular shape having an opposite connecting end and a loading end, the opposite connecting end being coupled to an end of the conduit, the guide positioning mechanism entering into the loading mechanism from the loading end.

14. The medical guide positioning device according to claim 5, further comprising a conduit and a loading mechanism, the loading mechanism being a hollow tubular shape having an opposite connecting end and a loading end, the opposite connecting end being coupled to an end of the conduit, the guide positioning mechanism entering into the loading mechanism from the loading end.

15. The medical guide positioning device according to claim 6, further comprising a conduit and a loading mechanism, the loading mechanism being a hollow tubular shape having an opposite connecting end and a loading end, the opposite connecting end being coupled to an end of the conduit, the guide positioning mechanism entering into the loading mechanism from the loading end.

16. The medical guide positioning device according to claim 7, further comprising a conduit and a loading mechanism, the loading mechanism being a hollow tubular shape having an opposite connecting end and a loading end, the opposite connecting end being coupled to an end of the conduit, the guide positioning mechanism entering into the loading mechanism from the loading end.

17. The medical guide positioning device according to claim 11, wherein a notch is opened in a side wall between the opposite connecting end and the loading end, the notch being configured to support passage of the extension portion.

18. The medical guide positioning device according to claim 12, wherein a notch is opened in a side wall between the opposite connecting end and the loading end, the notch being configured to support passage of the extension portion.

19. The medical guide positioning device according to claim 13, wherein a notch is opened in a side wall between the opposite connecting end and the loading end, the notch being configured to support passage of the extension portion.

20. The medical guide positioning device according to claim 14, wherein a notch is opened in a side wall between the opposite connecting end and the loading end, the notch being configured to support passage of the extension portion.
